(19) United States
(12) Patent Application Publication
(10) Pub. No.: US 2026/0345964 A1
(43) Pub. Date: Nov. 5, 2026

(54) MICROCATHETER

(71) Applicant: SHANGHAI MICROPORT ROTAPACE MEDTECH CO., LTD., Shanghai (CN)

(72) Inventors: Chen LIU, Shanghai (CN); Aiyang ZHANG, Shanghai (CN); Ruihui ZHAO, Shanghai (CN); Bin YUE, Shanghai (CN)

(21) Appl. No.: 19/121,236

(22) PCT Filed: Sep. 7, 2023
(86) PCT No.: PCT/CN2023/117449

§ 371 Date: Apr. 15, 2025

(30) Foreign Application Priority Data
Nov. 8, 2022 (CN) 202211392863.9

(51) Int. Cl. A61M 25/09; A61M 25/00

(52) U.S. Cl. CPC A61M 25/09041; A61M 25/0021; A61M 25/0045; A61M 25/005; A61M 25/0068; A61M 2025/0042

(57) ABSTRACT

The present invention relates to a microcatheter including a catheter body. The catheter body includes a tubular body and a conical tip disposed at a distal end of the tubular body. The tubular body includes an inner tube, a plurality of intermediate tubes and an outer tube, which are sequentially nested one inside another in this order from the inside outwards. Each of the inner tube and the outer tube is made of a polymeric material, and each of the intermediate tubes is made of a metallic material. This microcatheter exhibits enhanced torquability and can provide greater backup support, which impart effectively improved ability to handle a complex lesion.

### TECHNICAL FIELD

The present invention relates to the field of medical devices and, in particular, to a microcatheter with facilitated guidewire placement and exchanges.

### BACKGROUND

Percutaneous coronary intervention (PCI) is used primarily to treat obstructive coronary artery disease (CAD) and has shown rapid development thanks to its advantages including minimal invasion, time savings, high safety and high efficiency. According to statistics, there were as many as 1.224 million PCI procedures throughout China in 2020, representing a compound annual growth rate of as high as 16.3%. The market size of PCI-related interventional devices has reached 6.9 billion RMB yuan. Further, PCI has high success and good efficacy in the treatment of left main stem disease, chronic total occlusion (CTO), severe calcification and so forth.

Microcatheters can be used to assist guidewire placement and exchanges in peripheral and coronary vasculatures and inject contrast media and embolic or therapeutic agents and have been recognized as the first choice for the treatment of various coronary artery anomalies. CTO accounts for about 15% of PCI procedures, and almost all CTO procedures require the use of microcatheters. Clinically, microcatheters are desired to provide good backup support, torquability, pushability and crossability of the tip. However, currently available microcatheters tend to have poor torquability and inadequate backup support and pushability, which may lead to an extended period of time required by a surgical procedure using such a microcatheter, or even failure of the procedure. In addition, they tend to encounter significant resistance when the tip is being advanced through a guide catheter or blood vessel and cause some damage thereto, and have unsatisfactory crossability. Furthermore, their proximal design is uncomfortable and inconvenient for physicians, affecting the efficiency of their surgical operations.

### SUMMARY

It is an object of the present invention to provide a microcatheter, which overcomes at least one of the above-described problems with the conventional microcatheter products.

To this end, the present invention provides a microcatheter including a catheter body. The catheter body includes a tubular body and a conical tip disposed at a distal end of the tubular body. The tubular body includes an inner tube, a plurality of intermediate tubes and an outer tube, which are sequentially nested one inside another in this order from the inside outwards. Each of the inner tube and the outer tube is made of a polymeric material, and each of the intermediate tubes is made of a metallic material.

In one embodiment, the conical tip may define a guidewire lumen therein and the tubular body may define another guidewire lumen therein, which is in communication with and eccentric with respect to the guidewire lumen in the conical tip.

In one embodiment, an outer circumference of the conical tip may be coaxial with an outer circumference of the tubular body and eccentric with respect to the guidewire lumen in the conical tip. Alternatively, the outer circumference of the conical tip may be eccentric with respect to the outer circumference of the tubular body and concentric with the guidewire lumen in the conical tip.

In one embodiment, the guidewire lumen in the conical tip may be offset by 0.04-0.06 mm from the guidewire lumen in the tubular body.

In one embodiment, the conical tip may be non-linearly tapered. Additionally or alternatively, a helix may be provided over the outer circumference of the conical tip so as to be helically wound around the outer circumference of the conical tip along an axis thereof.

In one embodiment, a wire may be helically wound around the outer circumference of the outer tube along an axis thereof.

In one embodiment, the conical tip may include at least one of:
lubricious coatings on its edge portions;
filleted edges;
gel-like protective films wrapping its edge portions; and
an endothelialization coating on at least one of its inner and outer surfaces.

In one embodiment, the intermediate tubes may be coil spring(s) and/or braided tube(s).

In one embodiment, the intermediate tubes may include at least one coil spring and one braided tube.

In one embodiment, the intermediate tubes may be made of a nickel-titanium alloy material.

In one embodiment, the microcatheter may further include a coupling member shaped in conformity with the natural geometry of a hand and coupled to a proximal end of the tubular body.

In one embodiment, the coupling member may define depressions for facilitating gripping by the hand, the depressions provided with anti-slip features.

This microcatheter of the present invention offers at least the advantages as follows:

Through arranging a plurality of metallic intermediate tubes between the inner and outer tubes, the microcatheter shows remarkably improved overall torquability and can provide greater backup support. For some surgical procedures for treating a complex lesion, the enhanced torquability and backup support mean greatly improved ability of the microcatheter to handle the complex lesion. Thus, the microcatheter offers a wide range of benefits, including efficient passage through tortuous and spastic blood vessels enabling a surgical procedure using the microcatheter to be completed within a shorter period of time at lower cost and eliminating patients' pain and suffering, and can address various therapeutic needs.

### BRIEF DESCRIPTION OF THE DRAWINGS

Those of ordinary skill in the art will understand that the following drawings are presented to enable a better understanding of the present invention and not intended to limit the scope thereof in any sense, in which:

FIG. 1 is a schematic diagram showing the overall structure of a microcatheter according to an embodiment of the present invention;

FIG. 2 shows an axial cross sectional view of a tubular body of a microcatheter according to an embodiment of the present invention;

FIG. 3 is a schematic diagram showing the structure of a linearly conical tip according to an embodiment of the present invention;

FIG. 4 is a schematic diagram showing the structure of a non-linearly conical tip according to an embodiment of the present invention;

FIG. 5 is a schematic diagram showing the structure of a conical tip with a helix being wound around an outer circumference thereof according to an embodiment of the present invention;

FIG. 6 is a schematic diagram showing the structure of an outer tube in a tubular body with a wire being helically wound around an outer circumference thereof according to an embodiment of the present invention;

FIG. 7 is a schematic diagram showing the structure of an eccentric arrangement of a conical tip and a tubular body according to an embodiment of the present invention, in which an outer circumference of the conical tip is coaxial with an outer circumference of the tubular body, and a guidewire lumen section in the conical tip is not coaxial with a guidewire lumen section in the tubular body;

FIG. 8 is a schematic diagram showing the structure of an eccentric arrangement of a conical tip and a tubular body according to an embodiment of the present invention, in which an outer circumference of the conical tip is not coaxial with an outer circumference of the tubular body, and a guidewire lumen section in the conical tip is coaxial with a guidewire lumen section in the tubular body;

FIG. 9 depicts an application scenario of an eccentric arrangement of a conical tip and a tubular body according to an embodiment of the present invention, in which a guidewire is inserted and placed in a branch blood vessel;

FIG. 10 also depicts the application scenario, in which the conical tip in the eccentric arrangement is pivoted by an angle as a result of rotating a microcatheter so that the guidewire is inserted and placed in another branch blood vessel;

FIG. 11 shows a transverse cross-section of a tubular body according to an embodiment of the present invention, in which a coil spring is made from multiple wires;

FIG. 12 is a side view of a coupling member according to an embodiment of the present invention;

FIG. 13 is a font view of a coupling member according to an embodiment of the present invention;

FIG. 14 shows overall push force measurements of microcatheters according to embodiments of the present invention, in which the ordinate is the push force (in gf);

FIG. 15 shows distal rotation angle measurements of microcatheters according to embodiments of the present invention, in which the ordinate is the distal rotation angle (in °);

FIG. 16 shows backup support measurements of conical tips according to embodiments of the present invention, in which the ordinate is backup support (in N) and the abscissa is the distance from a distal end face of the conical tip (in mm); and

FIG. 17 shows crossability measurements of conical tips according to embodiments of the present invention, in which the ordinate is an advancement distance in a lesion (in mm).

### LIST OF REFERENCE NUMERALS

1 catheter body; 11 tip; 111 helix; 112 guidewire lumen; 12 tubular body; 121 inner tube; 122 intermediate tube; 122a coil spring; 122b braided tube; 123 outer tube; 124 wire; 2 coupling member; 21 depression; 22 anti-slip feature; 20 guidewire; 30 branch blood vessel.

### DETAILED DESCRIPTION

The present invention will be described in detail below with reference to the accompanying drawings, which show particular embodiments thereof. From the following description, advantages and features of the present invention will be more apparent. Note that the figure is provided in a very simplified form not necessarily drawn to exact scale for the only purpose of helping to explain the disclosed embodiments in a more convenient and clearer way. In order that the objects, features and advantages of the present invention can be more apparent and readily understood, reference is to be made to the accompanying drawings. It would be recognized that architectural, proportional, dimensional and other details in the figures are presented only for the purpose of facilitating, in conjunction with the disclosure herein, the understanding and reading of those familiar with the art, rather than being intended to limit conditions under which the present invention can be implemented. Therefore, any and all architectural modifications, proportional variations or dimensional changes that achieve the same or similar benefits and objects as the present invention are considered to fall within the scope of the teachings herein.

It is to be noted that, as used herein, relational terms such as first and second, etc., are only used to distinguish one entity or operation from another entity or operation, and do not necessarily require or imply these entities having such an order or sequence. Moreover, the terms “comprise,” “include,” or any other variations thereof are intended to cover a non-exclusive inclusion within a process, method, article, or apparatus that includes a list of elements including not only those elements but also those that are not explicitly listed, or other elements that are inherent to such processes, methods, goods, or equipment. In the case of no more limitation, the element defined by the sentence “includes a . . . ” does not exclude the existence of another identical element in the process, the method, or the device including the element.

Reference throughout this specification to “one embodiment”, “some embodiments”, “an example”, “a specific example” “some examples” or the like means that a particular feature, structure, material, or characteristic described in connection with the embodiments or examples is included in at least one embodiment or example of the invention. Thus, the appearances of those phrases in various places throughout this specification are not necessarily referring to the same embodiment or example of the invention. Furthermore, the particular features, structures, materials, or characteristics may be combined in any suitable manner in any one or more embodiments. Further, should there be no contradiction, one of ordinary skill in the art can combine the various embodiments, examples and features thereof described herein in any combination. Furthermore, in the following, for ease of illustration, the terms “axial” and “circumferential” may be used to describe directions along and around a central axis of a microcatheter, respectively.

In principle, the present invention seeks to provide a microcatheter suitable for use to assist guidewire placement and exchanges in peripheral and coronary vasculatures and inject contrast media and embolic or therapeutic agents. Preferably, it is suitable for use in percutaneous coronary intervention (PCI).

The proposed microcatheter exhibits better pushability, backup support, torquability and crossability than currently available microcatheter products, which effectively enhance its ability to handle complex lesions. During vascular intervention, the microcatheter can be advanced through a guide catheter into a target lumen to deliver functions, such as guidewire placement and exchanges, thanks to its excellent performance, which enable smooth performance of later surgical operations.

The present invention will be described in greater detail below with reference to the annexed figures, which show preferred embodiments thereof. Should there be no contradiction, the following embodiments and features thereof can be combined in any combination.

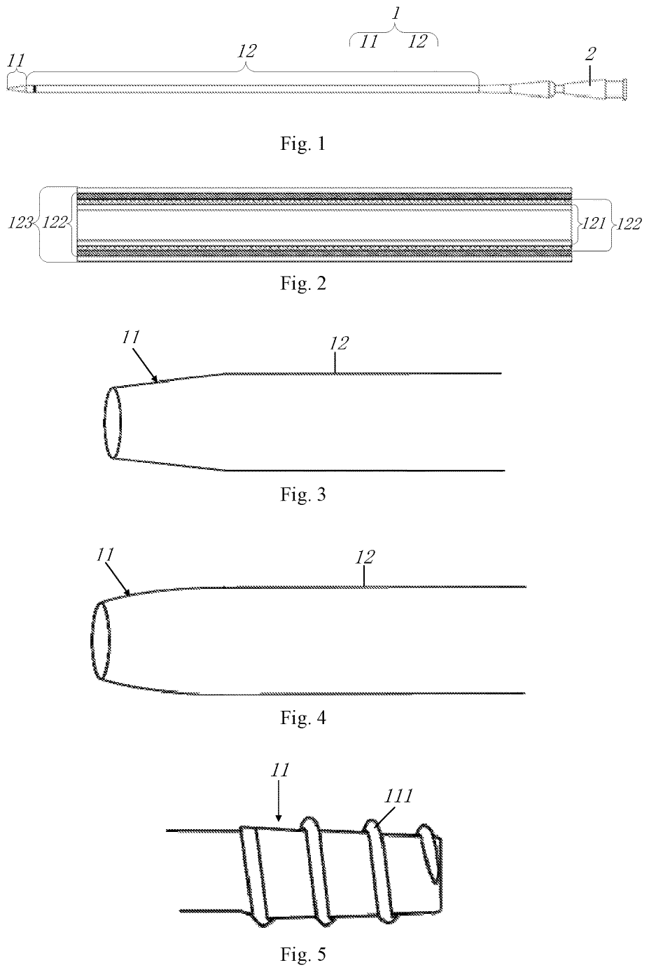

Reference is now made to FIG. 1, a schematic diagram showing the overall structure of a microcatheter according to an embodiment of the present invention. As shown in FIG. 1, the microcatheter defines a guidewire lumen, which extends through the microcatheter along its axis and is adapted for passage of a guidewire therethrough.

In this embodiment, the microcatheter includes a catheter body 1. The catheter body 1 includes a conical tip 11 and a tubular body 12. The conical tip 11 is provided at a distal end of the tubular body 12 and is fixedly coupled to the tubular body 12. The conical tip 11 and the tubular body 12 may be integrally formed, or made as separate pieces. Preferably, the conical tip 11 and the tubular body 12 are integrally formed, in order to ensure strong adhesion to each other. The tubular body 12 functions to ensure desirable overall backup support, torquability and pushability of the microcatheter, while the conical tip 11 serves to ensure desirable crossability of the distal end of the microcatheter and to position it at a target site in a blood vessel. The conical tip 11 and the tubular body 12 work with each other to ensure good overall performance of the microcatheter during use.

As shown in FIG. 2, in order to provide desirable backup support, torquability and pushability of the tubular body 12, the tubular body 12 is designed to include an inner tube 121, intermediate tubes 122 and an outer tube 123, which are nested one inside another sequentially in this order from the inside outwards. A plurality of intermediate tubes 122 are provided, e.g., two or more, preferably two or three intermediate tubes. All the intermediate tubes 122 may also be sequentially nested one inside another. The intermediate tubes 122 may be all of the same structure, or not. Here, by “of the same structure”, it is intended to means that they are identical in construction and/or material.

Each of the inner tube 121 and the outer tube 123 is made of a polymer material. Various polymeric materials are suitable, with those having good biocompatibility and good lubricity being particularly preferred. Considering that the outer tube 123 is to be brought into direct contact with the wall of a blood vessel or guide catheter, it is desired to be strong enough to provide adequate crush resistance of the outer tube 123. Accordingly, the outer tube 123 may be made of a polymeric material with high strength. Examples of this material may include, but are not limited to, polyether block amide (PEBA) (e.g., Pebax ®), thermoplastic polyurethane (TPU) elastomers, and combinations thereof. Considering that a guidewire will come into direct contact with an inner wall of the inner tube 121 (i.e., of the guidewire lumen), the inner wall of the inner tube 121 is desired to provide good lubricity, in order to reduce friction between it and the guidewire after they come into contact. Accordingly, the inner tube 121 may be made of a polymeric material with good lubricity. Examples of this material may include, but are not limited to, polytetrafluoroethylene (PTFE), polyvinyl chloride (PVC), polyether block amide (Pebax), high-density polyethylene (HDPE), nylon and combinations thereof.

Each intermediate tube 122 is made of a metallic material. Various metallic materials for medical use are suitable, with those with elasticity being preferred. Examples of the metallic material may include, but are not limited to, stainless steel and nickel-titanium alloys. In this embodiment, the material for forming the intermediate tubes 122 may be chosen as one or a combination of metallic materials with good strength and elasticity, such as stainless steel and nickel-titanium alloys, which can ensure desirable overall compliance of the microcatheter. Through incorporating the plurality of intermediate tubes 122, the microcatheter can provide greatly enhanced overall torquability and backup support. Moreover, the inner tube 121 allows easier passage of a guidewire therethrough with reduced resistance, and the outer tube 123 enables the microcatheter to be more easily pushed through a guide catheter or blood vessel and encounter less resistance. Each intermediate tube 122 may be implemented as a coil spring, a braided tube, or a combination thereof.

The intermediate tubes 122 may be all provided as coil springs, or braided tubes. Alternatively, at least one of the intermediate tubes 122 may be each provided as a coil spring, while at least one of the other intermediate tube(s) 122 may be each provided as a braided tube. Preferably, in order to allow the tubular body 12 to provide better backup support, pushability and torquability, the intermediate tubes 122 may include at least one braided tube and one coil spring. Since braided tubes provide better backup support, pushability and torquability than coil springs, the use of one or more coil springs in combination with one or more braided tubes can impart effectively improved torquability, backup support and pushability to the microcatheter. As would be appreciated, each coil spring, as well as each braided tube, may be made of one or more Grade 1 wires in any suitable shape, such as round or flat.

With this arrangement, the proposed microcatheter can provide remarkably improved overall torquability and increased backup support, which greatly enhance its ability to handle complex lesions in surgical applications. The microcatheter offers a wide range of benefits, including efficient passage through tortuous and spastic blood vessels, enabling a surgical procedure using the microcatheter to be completed within a shorter period of time at lower cost and eliminating patients' pain and suffering, and can address various therapeutic needs.

It will be recognized that the conical tip 11 may have an axial length as need.

As shown in FIG. 3, in some embodiments, the conical tip 11 is a linear cone, that is, the conical tip 11 is defined by a set of straight line segments. In these cases, the conical tip 11 may have a constant taper. When the conical tip 11 is a linear cone, the axial length of the conical tip 11 ranges from 0.5 mm to 10.0 mm and has a taper in the range of 0.175 to 0.195. The taper is determined by the axial length and an outer diameter of the conical tip 11. Here, the taper is defined as the ratio of half a diameter difference between distal and proximal end faces of the conical tip 11 to the axial length of the conical tip 11.

As shown in FIG. 4, in some embodiments, the conical tip 11 is a non-linear cone, that is, the conical tip 11 is defined by a set of curved line segments. In these cases, the conical tip 11 may have a varying taper. Compared with the linear cone, the non-linear cone can impart improved guiding properties and crossability to the conical tip 11 and avoid a sharp proximal edge of the conical tip 11 where it is joined to the tubular body 12. This effectively reduces damage possibly caused by the microcatheter to the wall of a blood vessel and enhances the overall pushability of the microcatheter and degree of fit with a blood vessel. When the conical tip 11 is non-linear, the curvature of the conical tip 11 (determined by angles between tangents and normal lines at its opposite ends) may range from 0.087 rad to 0.44 rad, in order to allow the conical tip 11 to be well buffered when advanced into a blood vessel while not causing any damage to the inner wall of the blood vessel due to excessive bulging. Compared with the linear cone, the non-linear cone enables higher safety and success of a surgical procedure using the microcatheter. When the conical tip 11 is the non-linear cone, its axial length and taper are also comparable to the conical tip 11 that is the linear cone.

The dimensions of the conical tip 11 (taper, outer diameter and axial length) may be determined depending on those of an associated guide catheter and a blood vessel to be intervened to ensure that the conical tip 11 possesses the most suitable guiding properties. Additionally, a total length of the conical tip 11 may be determined by taking into account its taper, in order to improve its guiding and other properties while ensuring the consistency and integrity of the conical tip 11 with the tubular body 12. For example, the conical tip 11 may have a maximum outer diameter (at its proximal end) of 0.4 mm to 0.6 mm, more preferably 0.4 mm to 0.5 mm, which can effectively enhance its pushability and crossability. Compared with the prior art, the conical tip 11 of the present invention is allowed to have a smaller outer diameter, which imparts better guiding properties to the microcatheter and enables it to smoothly pass through narrow blood vessels.

As shown in FIG. 5, in one embodiment, a helix 111 is provided on the outer circumference of the conical tip 11, the helix 111 is wound around an outer circumference of the conical tip 11 along an axis of the conical tip 11. The conical tip 11 may be made as an integral one-piece component. In this case, the helix 111 may integrally formed on the outer circumference of the conical tip 11 during its fabrication. Alternatively, the conical tip 11 may also be fabricated as a two-piece component. In this case, the conical tip 11 may be fabricated first, and the helix 111 may be then separately wound around the conical tip 11. Compared with the fabrication of the two-piece component, which additionally involves winding the helix 111 around the conical tip 11 and fixing them to each other, the fabrication of the one-piece component does not require such an assembly process and enables simpler fabrication. The helix 111 may have a pitch of 5.0-9.0 mm, and the helix 111 has an outer diameter slightly larger than that of the tubular body 12. For example, the outer diameter of the helix 111 may be 0.05-0.2 mm larger than that of the tubular body 12. Alternatively, the outer diameter of the helix 111 may be equal to that of the tubular body 12. Adding the helix 111 to the conical tip 11 can not only enhance its crossability and pushability, but also allows the microcatheter to be advanced in a guide catheter or blood vessel in different modes. For example, it may be screwed, instead of being pushed, through the guide catheter or blood vessel. This advancing mode is less invasive and better compatible with the tortuous nature of the coronary blood vessels. Thus, the microcatheter can be more easily advanced through tortuous blood vessels with different geometries and patterns of tortuosity, while showing improved pushability. Further, compared with separately fabricating the conical tip 11 and the helix 111 and then assembling them together, integrally forming them can reduce the chance of the helix 111 accidentally falling off the conical tip 11 in a blood vessel due to insecure attachment.

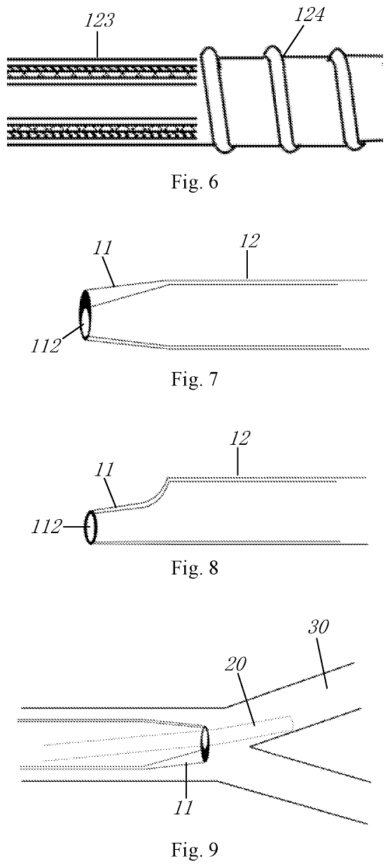

As shown in FIG. 6, in one embodiment, a wire 124 is helically wound around an outer circumference of the outer tube 123 along an axis thereof. Thus, the wire 124 is provided as another helix wound around the outer circumference of the outer tube 123. The wire 124 may have a diameter of 0.05-0.2 mm and a pitch 2-8 times the diameter. This arrangement is advantageous in that, in addition to being axially pushed, the microcatheter can also be screwed forward within blood vessels having different levels of tortuosity with an increased degree of fit therewith, which can reduce damage possibly caused by the microcatheter to the blood vessels. The wire 124 may be made of a polymeric or metallic material with high strength. The entire length or only part of the outer circumference of the outer tube 123 is wound with the wire 124. Therefore, adding the wire 124 can increase the torquability of the microcatheter and enables it to be screwed forward. The polymeric material of the wire 124 may be at least one of polyester, polyamide, PVC and a nylon elastomer. The metallic material of the wire 124 may be stainless steel, a nickel-titanium or other alloy, or a combination thereof.

In some embodiments, the structure of the conical tip 11 is friendly to guide catheters and blood vessels. Since the conical tip 11 is the component that is inserted into a guide catheter or blood vessel first, it is required to provide good guidance into the guide catheter or blood vessel, a low coefficient of friction and an appropriate degree of hardness or softness.

In one embodiment, lubricious coatings are applied to edge portions of the conical tip 11 (essentially, an edge portion around its proximal end and/or an edge portion at its distal end). The lubricious coatings may be coated with polyvinyl pyrrolidone (PVP), PTFE, parylene or the like, so as to cover the edge portions of the conical tip 11 with the lubricious coatings. The lubricious coatings can reduce friction between the conical tip 11 and a blood vessel or guide catheter and sharpness of the edge portions, thus reducing damage possibly caused by the conical tip 11 to the wall of the blood vessel and increasing safety and success of a surgical procedure using the microcatheter.

In one embodiment, the conical tip 11 is smooth but not sharp at the edges. These smooth edges can be typically obtained by filleting. For example, the proximal edge of the conical tip 11 (at its joint with the tubular body 12) is filleted and the distal edge of the conical tip 11 is filleted.

In one embodiment, the edge portions of the conical tip 11 is coated with gel-like protective films, which increase accessibility of the microcatheter. The protective films may be formed of a biological grade plastic gel-like material. Coating the edge portions of the conical tip 11 with the protective films can smooth the sharp edges.

The conical tip 11 and the tubular body 12 define the guidewire lumens, and the guidewire lumen 112 in the conical tip 11 is in communication with the guidewire lumen in the tubular body 12. Preferably, the guidewire lumen 112 in the conical tip 11 is eccentric with respect to the guidewire lumen in the tubular body 12. That is, a central axis of the guidewire lumen 112 in the conical tip 11 is not collinear with a central axis of the guidewire lumen in the tubular body 12, as particularly shown in FIGS. 7 to 8. This arrangement improves torquability of the conical tip 11, increases its selectivity to a blood vessel extending in a particular direction and allows it to be more easily positioned at a target site in the blood vessel. Thus, the microcatheter can be more quickly and more accurately directed toward a target lumen (e.g., a branch blood vessel, or a parent blood vessel) during a surgical procedure, simplifying operations involved in the surgical procedure, reducing risks associated with the procedure and increasing its safety. It will be understood that this eccentric arrangement can enhance torquability of the conical tip 11, and the offset between the two axes may be designed to attain selectivity to a target diseased blood vessel extending in a particular direction, significantly helping locating of the diseased blood vessel.

Preferably, the section of the guidewire lumen 112 in the conical tip 11 is offset by 0.04-0.06 mm from the section of the guidewire lumen in the tubular body 12. With this arrangement, although the section of the guidewire lumen 112 in the conical tip 11 is narrower, it still allows the passage of a 0.014″ guidewire. More importantly, the guidewire can be slightly deflected into an intended branch blood vessel to treat a target lesion therein. Limited by an outer diameter of a guidewire to be passed through the conical tip 11, an excessive offset may reduce an inner diameter of the conical tip 11 (i.e., of the section of the guidewire lumen 112 therein) to a value that disallows passage of the guidewire, while an inadequate offset may make it impossible for the guidewire to deflect into a branch blood vessel of interest due to an inadequate amount of deflection or an inappropriate angle of deflection.

As shown in FIG. 7, in one embodiment, the outer circumference of the conical tip 11 is coaxial with the outer circumference of the tubular body 12, the guidewire lumen 112 in the conical tip 11 is eccentric with respect to the outer circumference of the conical tip 11. In this case, the outer circumference of the conical tip 11 is coaxial with the outer circumference of the tubular body 12, the guidewire lumen 112 in the conical tip 11 is not coaxial with respect to the outer circumference of the conical tip 11.

As shown in FIG. 8, in an alternative embodiment, the outer circumference of the conical tip 11 is eccentric with respect to the outer circumference of the tubular body 12, the guidewire lumen 112 in the conical tip 11 is concentric with the outer circumference of the conical tip 11. In this case, the outer circumference of the conical tip 11 is not coaxial with the outer circumference of the tubular body 12, the guidewire lumen 112 in the conical tip 11 is coaxial with respect to the outer circumference of the conical tip 11.

Compared with the embodiment of FIG. 8, the embodiment of FIG. 7 is blood-vessel friendlier and associated with a lower probability of damage caused by the conical tip 11. However, in the embodiment of FIG. 8, the conical tip 11 can provide better guidance to a guidewire.

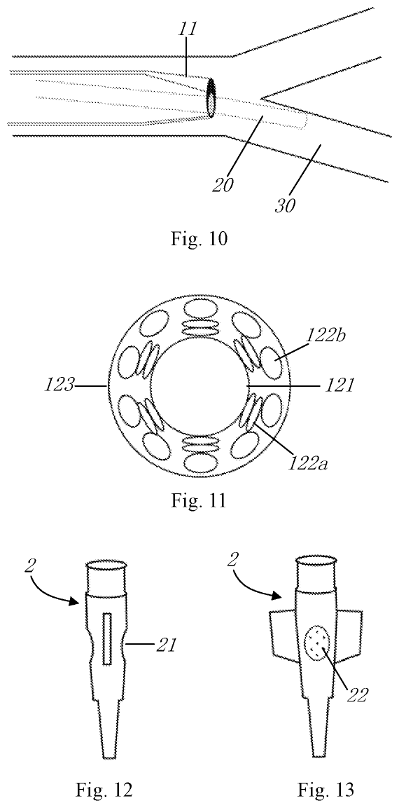

For example, the embodiment of FIG. 7 may be used in a blood vessel bifurcating into two branches, as shown in FIGS. 9 and 10. A guidewire 20 may be passed through the guidewire lumen of the microcatheter and protrude out of the distal end of the guidewire lumen 112 of the conical tip 11. As shown in FIG. 9, the guidewire 20 may be first extended into one of the branch blood vessels 30. After that, the microcatheter may be proximally rotated to alter the orientation of the guidewire lumen 112 in the conical tip 11 so that the guidewire 20 is pivoted into the other branch blood vessel 30. This arrangement allows the guidewire 20 to change its direction of distal advancement to accurately locate a target diseased blood vessel.

The conical tip 11 may be a composite structure consisting of two or more layers, which allows the conical tip 11 to exhibit appropriate overall hardness or softness, ensuring that the conical tip 11 can provide desirable backup support and flexibility. Here, by “a composite structure consisting of two or more layers”, it is intended to mean that the conical tip 11 is per se constructed from two or more tubes.

As the component of the microcatheter that comes into blood first, and as a foreign matter to the body, the conical tip 11 is desired to show good biocompatibility. Preferably, coatings capable of facilitating endothelialization are provided on inner and outer surfaces of the conical tip 11, in order to prevent blood coagulation on the surfaces. The coatings may be made of a material, such as heparin, clopidogrel bisulfate, aspirin, or a combination thereof, which can prevent platelet aggregation and blood coagulation.

The tubular body 12 is desired to be stiffer at its proximal end and less stiff at its distal end, in order to ensure desirable overall pushability and flexibility of the microcatheter. To this end, each intermediate tube 122 may be designed to have a stiffer proximal end and a less stiff distal end. For example, each intermediate tube 122 may have a stiffness profile gradually decreasing from the proximal end to the distal end. The present invention is not limited to any particular method for providing the decreasing stiffness profile. In practice, for example, one or more of the intermediate tubes 122 may each be provided as a coil spring having a varying pitch, which provides the stiffness profile. In this case, a more sparsely coiled spring portion possesses higher stiffness, and vice versa, i.e., a more densely coiled spring portion exhibits lower stiffness. Moreover, the tubular body 12 may have different turn pitches at different axial portions. Alternatively, one or more of the intermediate tubes 122 may each be provided as a braided tube having a varying wire density, which provides the stiffness profile. In this case, a tube portion with a higher wire density possesses higher stiffness, and vice versa, i.e., a tube portion with a lower wire density exhibits lower stiffness. Moreover, the tubular body 12 may have different wire densities at different axial portions. The wire densities may be measured in picks per inch, abbreviated as PPI. In addition, the backup support of the microcatheter can be increased by making each coil spring with an increased number of wires. For example, one or more of the intermediate tubes 122 may each be provided as a coil spring made from one or more helically coiled multi-strand wires. Additionally or alternatively, the backup support of the microcatheter can be increased by making each braided tube with an increased number of braid wires. For example, one or more of the intermediate tubes 122 may each be provided as a braided tube made from one or more multi-strand wires.

In general, each coil spring has a turn pitch not exceeding 2 times the diameter of a wire, if the coil spring has a turn pitch exceeding 2 times the diameter of a wire, the overall backup support of the microcatheter may be significantly affected and reduced. Each coil spring may be made from 1-11 wires. This wire count range can provide desirable torquability and allows overall hardness of the microcatheter to lie within an appropriate range. A greater number of wires would lead to excessive hardness, which is detrimental to overall compliance.

In one embodiment, each braided tube may have a wire density of 100-250 PPI. A wire density exceeding 250 PPI would lead to reduced overall hardness of the microcatheter, which is detrimental to pushability and braiding process consistency. On the other hand, a wire density of lower than 100 PPI will lead to excessive overall hardness of the microcatheter, which tends to cause damage to a guide catheter or blood vessel and is detrimental to the ability of the catheter to track a guidewire.

As shown in FIGS. 2 and 11, in a specific embodiment, the tubular body 12 includes an outer tube 123, two intermediate tubes 122 and an inner tube 121. The inner tube 121 has a wall thickness of 0.015-0.03 mm, and the inner tube 121 is preferred to be a PTFE tube. If the wall thickness of the inner tube 121 is too small, e.g., smaller than the lower limit of the 0.015-0.03 mm range, the inner tube 121 may be locally worn open after multiple times of use for guidewire placement and exchanges because of being too thin, thus exposing the guidewire placed therein. When this happens, luminal occlusion may occur to the microcatheter, not only overall affecting its performance in every aspect but also making it difficult for the guidewire to pass through the microcatheter to deliver its intended functions. On the other hand, if the wall thickness of the inner tube 121 is too large, then at a given overall outer diameter of the microcatheter, wall thicknesses of the metal tubes (i.e., the intermediate tubes 122) may have to be reduced. As a consequence of this, the microcatheter may be not able to provide sufficient backup support and torquability. If a wall thickness of the outer tube 123 is reduced, the outer tube 123 will be associated with an increased risk of being worn open over time. If this takes place, the guidewire received therein may be exposed, causing greater damage to the wall of a blood vessel.

As an example, the wall thickness of the outer tube 123 may be 0.1-0.25 mm, and an overall wall thickness of the two intermediate tubes 122 may range from 0.03 mm to 0.09 mm. One of the intermediate tubes 122 may be a braided tube, and the other may be a coil spring. In the embodiment of FIG. 11, one of the intermediate tubes 122 is a coil spring 122a, and the other is a braided tube 122b. The coil spring 122a is made from two coiled wires. Increasing the number of wires in the coil spring 122a can not only ensure an overall constant pitch profile of the coil spring 122a but can also enable the microcatheter to provide desirable torquability and backup support. It will be recognized that, to a surgical procedure for treating a complex lesion, whether the catheter used can provide desirable backup support and torquability is crucial. The proposed microcatheter is able to provide good torquability and backup support, enabling more accurate and reliable operations and increasing success of such a surgical procedure.

As shown in FIG. 1, the proposed microcatheter further includes an ergonomically designed coupling member 2, the coupling member 2 is shaped in conformity with the natural geometry of a human hand. The proximal end of the tubular body 12 is coupled to the coupling member 2 to allow an operator to operate the microcatheter by manipulating the coupling member 2. The ergonomic design of the coupling member 2 can provide increased comfort and convenience to a physician who is operating the microcatheter.

As shown in FIGS. 12 and 13, the coupling member 2 may define depressions 21 complementary in shape to finger segments in a preferential gripping posture. The depressions 21 may extend around the entire circumference of the coupling member 2, or symmetrically and circumferentially around the coupling member 2. Preferably, surfaces of the depressions 21 are provided thereon with anti-slip features 22 capable of increasing friction, for example, the anti-slip features 22 are bumps, pits, anti-slip coatings or combinations thereof. The anti-slip features 22 can increase contact friction between fingers and the coupling member 2, preventing the microcatheter from slipping out of the operator's hand and reducing safety risks associated with a surgical procedure using the microcatheter. The coupling member 2 may also be mechanically configured to be movable with the tubular body 12, for example so that rotating the coupling member 2 can cause the microcatheter to rotate to change the orientation of the guidewire lumen 112 in the conical tip 11 to allow the microcatheter to be advanced in a different direction, or to allow the guidewire to extend in a different direction.

In order to further demonstrate advantages of microcatheters constructed in accordance with embodiments of the present invention in terms of crossability, torquability and backup support and crossability of the tip, microcatheter samples were prepared in accordance with the present invention, each including a braided tube and a coil spring as two intermediate tubes, and analyzed in terms of pushability, torquability, compliance and crossability by comparing them with comparative examples, which were microcatheter samples, each including a single intermediate tube in the form of a braided tube, or a densely coiled spring.

In each of the three groups of samples, 10 samples were tested, each once, for crossability by evaluating their pushability and retractability. Specifically, each sample was pushed a fixed distance at a fixed constant speed with the aid of a guide catheter within a blood vessel model supported on a plate and immersed in a water bath at 37 °C. In each test, a maximum push force used during the advancement and retraction was recorded for comparison. A larger push force meant greater resistance encountered during advancement and poorer pushability, and vice versa. In each of the three groups of samples, 10 samples were also tested, each once, for torquability. In each test, the microcatheter was distally fixed to a sensor-equipped circular disk graduated with angles and proximally to a rotary motor and proximally rotated a fixed number of revolutions at a fixed constant speed, and an angle, by which the distal end had rotated, was then recorded. The closer the angles that the microcatheter rotated by at the distal and proximal ends were to each other, the better the torquability of the microcatheter was indicated. In addition, in each of the three groups of samples, 10 samples were subjected to three-point flexural tests performed on a universal testing machine to evaluate the conical tip′ backup support. For each microcatheter, a fixed-length segment of its conical tip was fixed at both ends and pushed a fixed distance at a fixed constant speed at the middle downwards, and a maximum flexure force in this process was recorded. A larger flexure force indicated greater backup support that the microcatheter could provide. The conical tip of each sample was tested at every 5 cm from its distal end. Further, two groups of samples (10 samples in each group) were further tested for crossability of the conical tips of the sample microcatheters. Each sample was tested once in a blood vessel model with a simulated occluded lesion. In each test, the microcatheter was proximally rotated three revolutions and pushed 5 mm forward, and a distance that the conical tip 11 was advanced in the occluded lesion was measured and recorded. The closer this distance was to the distance that the microcatheter was proximally pushed, the better the crossability of the tip was indicated.

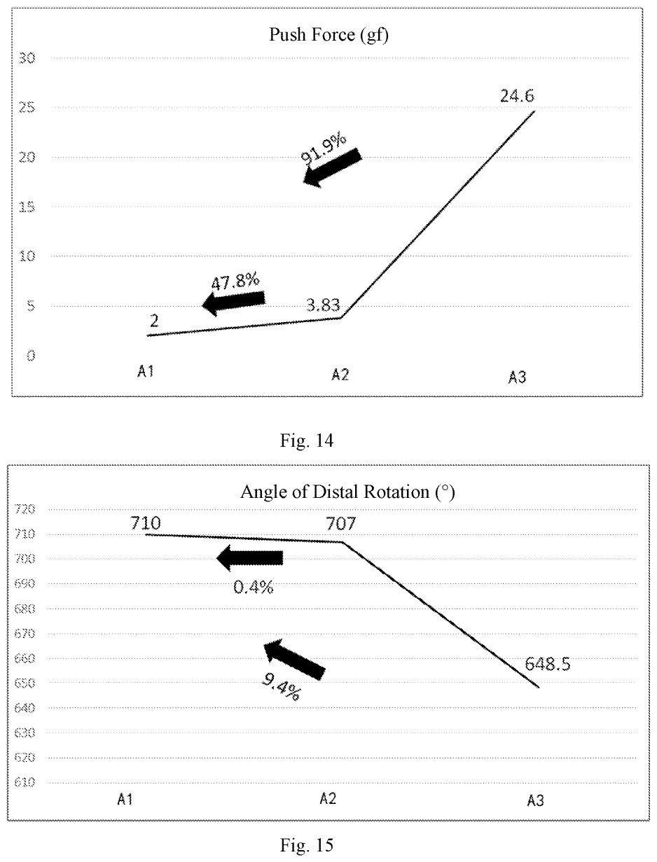

The results of the tests are presented in FIGS. 14 to 17, in which A1 represents the inventive samples each including two intermediate tubes, one of which is a braided tube, and the other is a coil spring; A2, Comparative Samples 1, each including a single intermediate tube in the form of a braided tube; A3, Comparative Samples 2, each including a single metal intermediate tube in the form of a coil spring; A4, smooth conical tips; and A5, conical tips each with a helix.

FIG. 14 shows the results of pushability tests. The push force of Comparative Samples 2 (A3) is 24.6 gf, the push force of Comparative Samples 1 (A2) is 3.83 gf and the push force of the inventive samples (A1) is 2 gf. According to these results, the inventive samples exhibit superior pushability because their push force is about 91.9% less than that of Comparative Samples 2 and about 47.8% less than that of Comparative Samples 1. Thus, the present invention is capable of significant improving pushability. As can be seen from the comparison of test data of FIG. 14, the microcatheter samples that each include both a braided tube and a coil spring as intermediate tubes can be pushed forward with a significantly reduced average push force, i.e., their pushability is significantly improved, compared to the other two types of microcatheters.

FIG. 15 shows the results of torquability tests, in which the ordinate is the angle of distal rotation in response to proximal rotation by 720°. The distal rotation angle of Comparative Samples 2 (A3) is 648.5°, the distal rotation angle of Comparative Samples 1 (A2) is 707°, and the distal rotation angle of the inventive samples (A1) is 710°. According to these results, the inventive samples exhibit superior torquability because their distal rotation angle is about 9.4% larger than that of Comparative Samples 2 and about 0.4% larger than that of Comparative Samples 1. Therefore, as can be seen from the comparison of test data of FIG. 15, the distal rotation angle of the microcatheter samples that each include both a braided tube and a coil spring as intermediate tubes is closer to the proximal rotation angle, indicating significantly improved torquability of them.

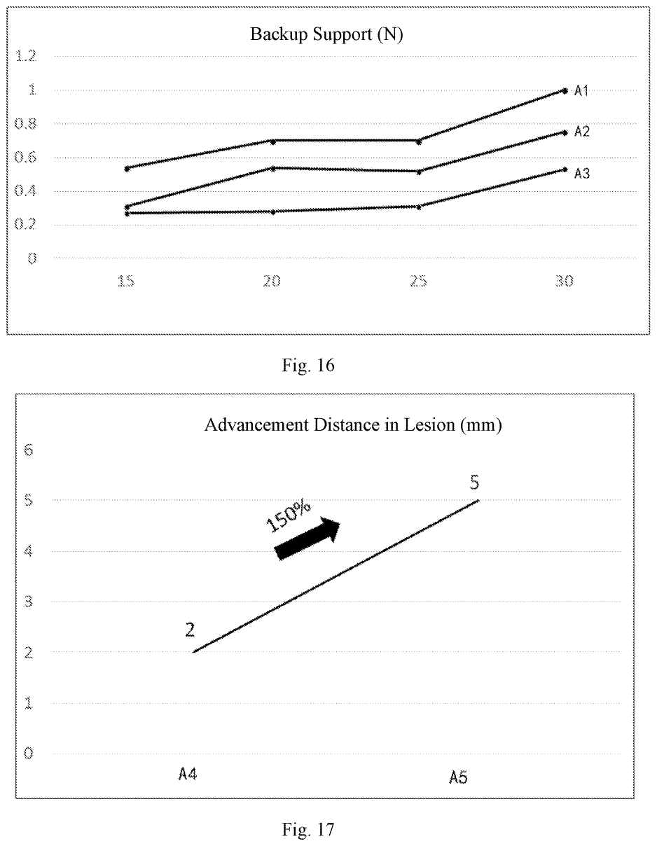

FIG. 16 shows the results of backup support tests on conical tips. Each conical tip 11 was 30 mm long, and measurements were taken at 15 mm, 20 mm, 25 mm and 30 mm away from the distal end face thereof. The tips of Comparative Samples 2 (A3) provide significantly less backup support than those of Comparative Samples 1 (A2) and the inventive samples (A1). The tips of the inventive samples A1 provide significantly greater backup support than those of Comparative Samples 1 (A2). Greater backup support means increased resistance to flexure. Therefore, as can be seen from the comparison of test data of FIG. 16, the conical tips 11 of the microcatheter samples that each include both a braided tube and a coil spring as intermediate tubes can provide greater backup support at a location 15-30 cm away from the distal end face, which imparts high crossability through a lesion, compared with the other two types of microcatheters.

FIG. 17 shows the results of tests on crossability of conical tips through a simulated 10-mm long lesion, in which A4 represents smooth conical tips, and A5 represents conical tips each with a helix. According to these results, the conical tips A5 each with a helix exhibit higher crossability than the smooth conical tips A4—the smooth conical tips A4 were advanced 2 mm, while the conical tips A5 were advanced 5 mm, in the lesion, representing an increase of 150% in crossability. As can be seen from the comparison of test data of FIG. 17, as the conical tips 11 each with a helix or helical structure can be screwed and advanced, they can be better passed through an occluded lesion and therefore have enhanced crossability.

In summary, according to the present invention, through arranging a plurality of metallic intermediate tubes 122 between the inner tube 121 and the outer tube 123, the proposed microcatheter shows remarkably improved overall torquability and can provide greater backup support. For some surgical procedures for treating a complex lesion, the enhanced torquability and backup support mean greatly improved ability of the microcatheter to handle the complex lesion. Thus, the proposed microcatheter offers a wide range of benefits, including efficient passage through tortuous and spastic blood vessels enabling a surgical procedure using the microcatheter to be completed within a shorter period of time at lower cost and eliminating patients' pain and suffering, and can address various therapeutic needs.

It is noted that many modifications and additions may be made by those of ordinary skill in the art without departing from the teachings disclosed hereinabove, and such modifications and additions are intended to be also included within the scope of the present invention. Any and all equivalent changes such as alternations, modifications and variations made in light of the above disclosure by those familiar with the art without departing from the spirit and scope of the invention are all considered as equivalent embodiments of the present invention. Accordingly, any and all equivalent changes such as alternations, modifications and variations made to the disclosed embodiments in accordance with the essential principles of the present invention fall within the scope of the invention.

1. A microcatheter, comprising a catheter body, the catheter body comprising a tubular body and a conical tip disposed at a distal end of the tubular body, the tubular body comprising an inner tube, a plurality of intermediate tubes and an outer tube, which are sequentially nested one inside another in this order from the inside outwards, each of the inner tube and the outer tube made of a polymeric material, each of the intermediate tubes made of a metallic material.

2. The microcatheter according to claim 1, wherein the conical tip defines a guidewire lumen therein and the tubular body defines another guidewire lumen therein, the guidewire lumen in the conical tip is in communication with the guidewire lumen in the tubular body and the guidewire lumen in the conical tip is eccentric with respect to the guidewire lumen in the tubular body.

3. The microcatheter according to claim 2, wherein an outer circumference of the conical tip is coaxial with an outer circumference of the tubular body and the guidewire lumen in the conical tip is eccentric with respect to the outer circumference of the conical tip, or wherein the outer circumference of the conical tip is eccentric with respect to the outer circumference of the tubular body and the guidewire lumen in the conical tip is concentric with the outer circumference of the conical tip.

4. The microcatheter according to claim 2, wherein the guidewire lumen in the conical tip is offset by 0.04-0.06 mm from the guidewire lumen in the tubular body.

5. The microcatheter according to claim 1, wherein the conical tip is non-linearly tapered and/or wherein a helix is provided over the outer circumference of the conical tip, the helix helically wound around the outer circumference of the conical tip along an axis of the conical tip.

6. The microcatheter according to claim 1, wherein a wire is helically wound around the outer circumference of the outer tube along an axis of the outer tube.

7. The microcatheter according to claim 1, wherein the conical tip comprises at least one of:
lubricious coatings on edge portions of the conical tip;
filleted edges on the edge portions of the conical tip;
gel-like protective films wrapping the edge portions of the conical tip; and
an endothelialization coating on at least one of an inner surface and an outer surface of the conical tip.

8. The microcatheter according to claim 1, wherein the intermediate tubes are coil spring(s) and/or braided tube(s).

9. The microcatheter according to claim 8, wherein the intermediate tubes comprise at least one coil spring and one braided tube.

10. The microcatheter according to claim 8, wherein the intermediate tubes are made of a nickel-titanium alloy material.

11. The microcatheter according to claim 1, further comprising a coupling member shaped in conformity with a natural geometry of a hand, and wherein a proximal end of the tubular body is coupled to the coupling member.

12. The microcatheter according to claim 11, wherein the coupling member defines depressions for facilitating gripping by the hand, and wherein the depressions are provided with anti-slip features.
